(19) United States
(12) Patent Application Publication
(10) Pub. No.: US 2022/0160682 A1
(43) Pub. Date: May 26, 2022

(54) SYSTEMIC ISOXAZOLINE PARASITICIDES FOR VECTOR-BORNE AND VIRAL DISEASE TREATMENT OR PROPHYLAXIS

(71) Applicant: Tarsus Pharmaceuticals, Inc., Irvine, CA (US)

(72) Inventors: Bobak Robert Azamian, Newport Coast, CA (US); Douglas Michael Ackermann, Reno, NV (US); Christopher William Stivers, Lynnfield, MA (US); Shawn D. Hickok, Aliso Viejo, CA (US)

(21) Appl. No.: 17/594,144

(22) PCT Filed: Apr. 3, 2020
(86) PCT No.: PCT/IB2020/053229

§ 371 Date: Oct. 4, 2021

Related U.S. Application Data
(60) Provisional application No. 62/829,573, filed on Apr. 4, 2019.

(51) Int. Cl. A61K 31/422; A61K 31/42; A61K 31/519; A61K 31/513; A61K 31/427; A61K 31/635; A61K 31/495; A61K 31/675; A61K 31/7056; A61K 31/4706; A61K 31/4709

(52) U.S. Cl. CPC A61K 31/422; A61K 31/42; A61K 31/519; A61K 31/513; A61K 31/427; A61K 31/4709; A61K 31/495; A61K 31/675; A61K 31/7056; A61K 31/4706; A61K 31/635

(57) ABSTRACT

Disclosed herein are methods of treating or preventing infections associated with organisms, or preventing vector-borne diseases including Plasmodium infestation and/or malaria via delivery of one, two, or more systemic doses of an isoxazoline anti-parasitic therapeutic agent to an individual with confirmed or suspected infestation of Plasmodium and/or malaria.

### PRIORITY CLAIM

This application claims the benefit under 35 U.S.C. § 119(e) of U.S. Prov. App. No. 62/829,573 filed on Apr. 4, 2019, which is hereby incorporated by reference in its entirety.

### BACKGROUND

In some aspects, embodiments of the invention relate to treating and preventing various vector-borne and other transmittable pathogenic organisms, of which new therapeutic and prophylactic modalities are sorely needed.

### SUMMARY

In some embodiments, disclosed herein are formulations, including pharmaceutical formulations, and methods of using the same for treatment and/or prophylaxis of a variety of vector-borne and other pathogenic diseases and related organisms, that comprise, consist essentially of, or consist of any number of features/elements disclosed herein.

In some embodiments, disclosed herein are formulations, including pharmaceutical formulations, and methods of using the same for treatment and/or prophylaxis of a variety of pathogenic diseases and related organisms involving parasites, bacteria, viruses, fungi, and/or other organisms that comprise, consist essentially of, or consist of any number of features/elements disclosed herein.

In some embodiments, disclosed herein is a method of treating malaria, comprising: administering a therapeutic dose of an isoxazoline parasiticide formulation therapeutically effective to an individual in need thereof, the therapeutic dose sufficient to be systemically bioavailable sufficient to inhibit the health or life cycle of a Plasmodium species in the individual.

In some embodiments, the method comprises administering a plurality of doses of the isoxazoline parasiticide formulation within a 30 day period.

In some embodiments, the formulation is administered orally.

In some embodiments, the formulation is administered parenterally.

In some embodiments, the formulation is administered transdermally.

In some embodiments, the Plasmodium species is selected from the group consisting of: P. falciparum, P. vivax, P. malaria, P. ovale, and P. knowlesi.

In some embodiments, the formulation is therapeutically effective to inhibit the health or life cycle of the Plasmodium species in a liver of the individual.

In some embodiments, the method further comprises administering another therapeutic agent therapeutically effective to inhibit the health or life cycle of a Plasmodium species in the individual.

In some embodiments, administering another therapeutic agent occurs in the same formulation as the isoxazoline parasiticide formulation.

10. The method of any of the preceding claims, further comprising identifying the individual diagnosed with malaria.

In some embodiments, the isoxazoline parasiticide is selected from the group consisting of fluralaner, lotilaner, sarolaner, and afoxolaner.

In some embodiments, disclosed is an isoxazoline parasiticide formulation for use in treating malaria, said formulation therapeutically effective to an individual in need thereof, said formulation sufficient to be systemically bioavailable sufficient to inhibit the health or life cycle of a Plasmodium species in the individual.

In some embodiments, the isoxazoline parasiticide is selected from the group consisting of fluralaner, lotilaner, sarolaner, and afoxolaner.

Also disclosed herein is a method of prophylaxis against a vector-borne disease, comprising: administering a single therapeutic dose of an isoxazoline parasiticide formulation therapeutically effective to an individual in need thereof, the single therapeutic dose sufficient to be systemically bioavailable sufficient to inhibit the health or life cycle of a vector or vector-borne disease organism for at least about 1 month.

In some embodiments, the method is sufficient to be systemically bioavailable sufficient to inhibit the health or life cycle of a vector or vector-borne disease organism for at least about 3 months.

In some embodiments, the isoxazoline parasiticide is selected from the group consisting of fluralaner, lotilaner, sarolaner, and afoxolaner.

In some embodiments, disclosed herein is a method of prophylaxis against a vector-borne disease, comprising: administering a plurality of spaced-apart therapeutic doses of an isoxazoline parasiticide formulation therapeutically effective to an individual in need thereof, wherein the spaced-apart therapeutic doses include between 2-7 doses within a week, but no further doses within at least about a 1 month period, the plurality of space-apart therapeutic doses sufficient to be systemically bioavailable sufficient to inhibit the health or life cycle of a vector or vector-borne disease organism for at least about 1 month.

In some embodiments, the plurality of spaced-apart therapeutic doses are oral doses of an isoxazoline parasiticides, the oral doses each about or less than about 500 mg.

In some embodiments, the method further comprises no further doses within at least about a 3 month period.

In some embodiments, the method is sufficient to be systemically bioavailable sufficient to inhibit the health or life cycle of a vector or vector-borne disease organism for at least about 3 months.

In some embodiments, the vector-borne disease comprises malaria.

In some embodiments, the vector-borne disease comprises Lyme disease.

In some embodiments, the vector-borne disease comprises one or more of the group consisting of: dengue, West Nile virus, chikungya, yellow fever, filiarisis, tularemia, dilofilariasis, Japanese encephalitis, St. Louis encephalitis, Western equine encephalitis, Zika, EEE (Eastern Equine Encephalitis), Lyme Disease, Anaplasmosis, Ehrlichiosis, Babesiosis, Borrelia miyamotoi disease, Rickettsia parkeri spotted fever, Pacific Coast tick fever, Ehrlichia muris-like infection, Heartland virus, Bourbon virus, B. mayonii infection, and other tickborne diseases.

In some embodiments, disclosed herein are methods of treating or preventing a viral infection, comprising: administering to a subject in need thereof a pharmaceutical composition comprising an isoxazoline parasiticide, the formulation therapeutically effective to treat or prevent the viral infection in the subject.

In some embodiments, the method comprises treating the viral infection.

In some embodiments, the pharmaceutical composition is a single one-time dose.

In some embodiments, the viral infection comprises a coronavirus infection.

In some embodiments, the viral infection comprises SARS-CoV 2 (COVID 19).

In some embodiments, the pharmaceutical composition is sufficient to be systemically bioavailable sufficient to inhibit the health or life cycle of the virus for at least about 1 month.

In some embodiments, the isoxazoline parasiticide is selected from the group consisting of: fluralaner, sarolaner, lotilaner, afoxolaner, fluxametamide, and isocycloseram.

In some embodiments, the isoxazoline parasiticide is the single active agent in the pharmaceutical composition.

In some embodiments, the method further comprises one or more of the following additional active agents: baricitinib; lopinavir and/or ritonavir, darunavir, favipiravir, remdesivir, ribavirin, galidseivir, BCX-4430, Arbidol, chloroquine, hydroxychloroquine, mefloquine, and/or nitazoxanide.

In some embodiments, disclosed herein is a method of prophylaxis against a viral infection, comprising: administering a plurality of spaced-apart therapeutic doses of an isoxazoline parasiticide formulation therapeutically effective to an individual in need thereof, wherein the spaced-apart therapeutic doses include between 2-7 doses within a week, but no further doses within at least about a 1 month period, the plurality of space-apart therapeutic doses sufficient to be systemically bioavailable sufficient to inhibit the life cycle and/or replication of a virus for at least about 1 month.

In some embodiments, the viral infection comprises a coronavirus.

In some embodiments, the viral infection comprises SARS-CoV 2 (COVID 19).

In some embodiments, the plurality of spaced-apart therapeutic doses are oral doses of an isoxazoline parasiticides, the oral doses each about or less than about 500 mg.

In some embodiments, the method further comprises no further doses within at least about a 3 month period.

In some embodiments, the method is sufficient to be systemically bioavailable sufficient to inhibit the replication or life cycle of a virus for at least about 3 months.

In some embodiments, the isoxazoline parasiticide is selected from the group consisting of: fluralaner, sarolaner, lotilaner, afoxolaner, fluxametamide, and isocycloseram.

In some embodiments, the method further comprises one or more of the following additional active agents: baricitinib; lopinavir and/or ritonavir, darunavir, favipiravir, remdesivir, ribavirin, galidseivir, BCX-4430, Arbidol, chloroquine, hydroxychloroquine, mefloquine, and/or nitazoxanide.

Also disclosed herein is an isoxazoline parasiticide medicament for use in treating or preventing a pathogen, said medicament therapeutically effective to an individual in need thereof, said formulation sufficient to be systemically bioavailable sufficient to inhibit the health or life cycle of the pathogen.

In some embodiments, the pathogen comprises a virus.

In some embodiments, the virus comprises a coronavirus.

In some embodiments, the virus comprises SARS-CoV 2 (COVID 19).

In some embodiments, the medicament is for treating the pathogen.

In some embodiments, the medicament is for preventing the pathogen.

In some embodiments, the isoxazoline parasiticide is selected from the group consisting of: fluralaner, sarolaner, lotilaner, afoxolaner, fluxametamide, and isocycloseram.

### DETAILED DESCRIPTION

Malaria is a serious, sometimes life-threatening disease caused by Plasmodium parasites that are transmitted to people through the bites of infected Anopheles mosquitoes, known as malaria vectors. According to the World Health Organization (WHO), in 2017, there were an estimated 219 million cases of malaria in 87 countries, and the estimated number of malaria deaths stood at 435,000 in 2017.

The WHO African Region is said to carry a disproportionately high share of the global malaria burden. In 2017, the region was home to 92% of malaria cases and 93% of malaria deaths. According to WHO, 5 countries accounted for nearly half of all malaria cases worldwide: Nigeria (25%), the Democratic Republic of the Congo (11%), Mozambique (5%), India (4%) and Uganda (4%). It has been estimated that nearly half of the world's population was at risk of malaria. Most malaria cases and deaths occur in sub-Saharan Africa. However, the WHO regions of South-East Asia, Eastern Mediterranean, Western Pacific, and the Americas are also at risk. In 2017, 87 countries and areas had ongoing malaria transmission.

Total funding for malaria control and elimination reached an estimated $3.1 billion in 2017. Some population groups are at considerably higher risk of contracting malaria, and developing severe disease, than others. These include infants, children under 5 years of age, pregnant women and patients with HIV/AIDS, as well as non-immune migrants, mobile populations and travelers.

There are at least 5 parasite species that cause malaria in humans, including P. falciparum, P. vivax, P. malaria, P. ovale, and P. knowlesi. In 2017, P. falciparum accounted for 99.7% of estimated malaria cases in the WHO African Region, as well as in the majority of cases in the WHO regions of South-East Asia (62.8%), the Eastern Mediterranean (69%) and the Western Pacific (71.9%). P. vivax is the predominant parasite in the WHO Region of the Americas, representing 74.1% of malaria cases.

Malaria is an acute, febrile illness. Symptoms typically manifest about 10-15 days after the culprit mosquito bite. The initial symptoms, typically fever, headache, and chills may be mild and difficult to recognize as malaria. If not treated within 24 hours, P. falciparum malaria for example can progress to severe illness, often leading to death.

Children with severe malaria frequently develop one or more of the following symptoms: severe anemia, respiratory distress in relation to metabolic acidosis, or cerebral malaria. In adults, multi-organ failure is also frequent. In malaria endemic areas, people may develop partial immunity, allowing asymptomatic infections to occur.

There are more than 400 different species of Anopheles mosquito; around 30 are malaria vectors of major importance. Typical vector mosquitoes bite at night time. The intensity of transmission depends on factors related to the parasite, the vector, the human host, and the environment.

Anopheles mosquitoes lay their eggs in water, which hatch into larvae, eventually emerging as adult mosquitoes. The female mosquitoes seek a blood meal to provide nutrition to their eggs. Transmission can be of increased incidence in locations where the mosquito lifespan is longer (so that the parasite has time to complete its development inside the mosquito) and where it prefers to bite humans rather than other animals. The long lifespan and strong human-biting habit of the African vector species is a primary reason why approximately 90% of the world's malaria cases are in Africa.

Transmission also depends on climatic conditions that may affect the number and survival of mosquitoes, such as rainfall patterns, temperature and humidity. In many places, transmission is seasonal, with the peak during and just after the rainy season. Malaria epidemics can occur when climate and other conditions suddenly favor transmission in areas where people have little or no immunity to malaria. They can also occur when people with low immunity move into areas with intense malaria transmission, for instance to find work, or as refugees.

Human immunity is another important factor, especially among adults in areas of moderate or intense transmission conditions. Partial immunity is developed over years of exposure, and while it never provides complete protection, it does reduce the risk that malaria infection will cause severe disease. For this reason, most malaria deaths in Africa occur in young children, whereas in areas with less transmission and low immunity, all age groups are at risk.

Due to at least the foregoing, improved systems and methods of treating and/or preventing malaria, as well as other conditions including those disclosed herein, are needed.

In some embodiments, disclosed herein are methods of treating Plasmodium infestation and/or malaria via delivery of one, two, or more systemic doses of an isoxazoline anti-parasitic therapeutic agent to an individual with confirmed or suspected infestation of Plasmodium and/or malaria.

In some embodiments, disclosed herein are methods of treating infestation of vector-borne organisms such as, for example, Borrelia Burgdorferi, Borrelia mayonii, Borrelia miyamotoi, other Borrelia species, Babesia microti, other Babesia species, Ehrlichia muris eauclairensis, Ehrlichia chaffeensis, Ehrlichia ewingii, other Ehrlichia species, Anaplasma phagocytophilum, other Anaplasma species, Francisella tularensis, other Francisella species, Rickettsia rickettsia, Rickettsia parkeri, other Rickettsia species, Powassan virus, Heartland virus, Bourbon virus, and/or Colorado tick fever virus via delivery of one, two, or more systemic doses of an isoxazoline anti-parasitic therapeutic agent to an individual with confirmed or suspected infestation of such vector-borne organisms and/or any disease resulting therefrom.

In some embodiments, disclosed herein are methods of preventing vector-borne disease in humans and other animals, such as, for example, Lyme Disease, Anaplasmosis, Ehrlichiosis, Babesiosis, Borrelia miyarnotoi disease, relapsing fever, Powassan virus disease, Tularemia, Heartland virus disease, Bourbon virus disease, Rocky Mountain spotted fever, R. parkeri rickettsiosis, Colorado tick fever, tick-borne relapsing fever, Southern tick-associated rash illness, or other such tick-borne diseases, via delivery of one, two, or more systemic doses of an isoxazoline anti-parasitic agent to an individual, and which prevents infection by killing the organism-carrying vector, for example mosquitoes of the Anopheles genus (including Anopheles gambiae, Anopheles stephensi, and others), Ixodes Scapularis, Amblyomma americanum, Dermacentor variabilis, Rhipicephalus sanguineus, Ixodes cookie, Amblyomma maculatum, Dermacentor andersoni, Ornithodoros spp., Ixodes pacificus, or other species of relevant vector, prior to passage of the organism from vector to human, or prior to biting another human in close proximity, for example within about 1, 2, 4, 6, 8, 12, 15, 18, 24 or more hours, within about 5, 10, 15, 30, 45 minutes, or in the range of about 4-8 hours), or ranges including any two of the foregoing values.

In some embodiments, disclosed herein are methods of preventing vector-borne disease in humans via delivery of one, two, or more systemic doses of an isoxazoline anti-parasitic agent to one or more individuals in close geographic proximity (for example, within about 1, 2, 3, 4, 5, 6, 7, 8, 9, 10 or more square miles, within about 10 to 50 square miles, or within about 100 square miles), and which prevents infection by killing disease-carrying vectors such as mosquitoes of the Anopheles genus (for example Anopheles gambiae, Anopheles stephensi, and others) after biting said individual(s) to thereby reduce the local vector population.

Also disclosed herein are isoxazoline parasiticide formulations for use in treating vector-borne diseases such as Lyme disease, Anaplasmosis, or malaria, or others as disclosed elsewhere herein, said formulation therapeutically effective to an individual in need thereof, said formulation sufficient to be systemically bioavailable sufficient to inhibit the health or life cycle of a vector-borne organism such as a Plasmodium species in the individual. The formulation could have any number of properties as disclosed elsewhere herein.

Also disclosed herein are isoxazoline parasiticide formulations for use in preventing vector-borne disease, said formulation sufficient to be systemically bioavailable sufficient to cause the death of a vector that encounters said systemically circulating anti-parasitic agent with reasonable probability (for example, more than about 50%, 60%, 70%, 80%, 90%, or more or less likelihood of vector mortality). The formulation could have any number of properties as disclosed elsewhere herein.

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formula, including the racemates, stereoisomers, and tautomers of the compound or compounds.

As used herein, the term “effective amount” means that amount of a drug or pharmaceutical agent that will elicit the biological or medical response of a tissue, system, human or non-human animal that is being sought, for instance, by a researcher or clinician. In some embodiments

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “excipient” means the substances used to formulate active pharmaceutical ingredients (API) into pharmaceutical formulations. Excipients (e.g., mannitol, Captisol®, lactose, starch, magnesium stearate, sodium saccharine, talcum, cellulose, sodium crosscarmellose, glucose, gelatin, sucrose, magnesium carbonate, and the like) are an integral part of pharmaceutical development and help to achieve the desired product profile including but not limited to an aid in manufacturing, modify a drug's stability, and efficacy. Acceptable excipients are non-toxic and do not adversely affect the therapeutic benefit of at least one chemical entity described herein. Such excipient may be any solid, liquid, semi-solid or, in the case of an aerosol composition, gaseous excipient that is generally available.

Further the term “excipient’ encompasses solubilizing agents, stabilizers, carriers, diluents, bulking agents, pH buffering agents, tonicifying agents, antimicrobial agents, wetting agents, and emulsifying agents (e.g., sodium acetate, sodium citrate, cyclodextrine derivatives, sorbitan monolaurate, triethanolamine acetate, triethanolamine oleate, and the like). Preferably, excipients are approved for or considered to be safe for human and other animal administration. Generally, depending on the intended mode of administration, the pharmaceutical composition will contain about 0.005% to 95%; in certain embodiments, about 0.5% to 50% by weight of a chemical entity. As used here in, “lyophilization”, “lyophilized,” and “freeze-dried” refers to a process by which the material to be dried is first frozen and then the ice or frozen solvent is removed by sublimation in a vacuum environment. The term “lyophilized powder” or “lyophilized preparation” refers to any solid material obtained by lyophilization, i.e., freeze-drying of an aqueous solution. The aqueous solution may contain non-aqueous solvents, i.e. a solution composed of aqueous and one or more non-aqueous solvent(s). Preferably, a lyophilized preparation is one in which the solid material is obtained by freeze-drying a solution composed of water as a pharmaceutically acceptable excipient.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate.

Pharmaceutically acceptable salts of compounds may be prepared. These pharmaceutically acceptable salts may be prepared in situ during the final isolation and purification of the compound, or by separately reacting the purified compound in its free acid or free base form with a suitable base or acid, respectively.

Accordingly, the word “or” in the context of “a compound or a pharmaceutically acceptable salt thereof is understood to refer to either a compound or a pharmaceutically acceptable salt thereof (alternative), or a compound and a pharmaceutically acceptable salt thereof (in combination).

As used herein, the term “pharmaceutical composition” (which can also be referred to herein as a formulation or formulations) describes a compound and one or more pharmaceutically acceptable excipients. The excipient(s) can be acceptable in the sense of being compatible with the other ingredients of the composition and not deleterious to the recipient thereof. In accordance with another aspect of the invention there is also provided a process for the preparation of a pharmaceutical composition including the agent, or pharmaceutically acceptable salts thereof, with one or more pharmaceutically acceptable excipients. The pharmaceutical compositions can be for use in the treatment and/or prophylaxis of any of the conditions described herein.

Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

“Racemates” refers to a mixture of enantiomers. In an embodiment of the invention, a therapeutic agent, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent.

Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

Optically active (R)- and (S)-isomers and d and I isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

Such compounds of some embodiments can exist in particular geometric or stereoisomeric forms. The invention contemplates all such compounds, including (−)- and (+)-enantiomers, (R)- and (S)-enantiomers, (D)-isomers, (L)-isomers, the racemic mixtures thereof, and other mixtures thereof, such as enantiomerically enriched mixtures, as falling within the scope of the invention. Additional asymmetric carbon atoms can be present in a substituent such as an alkyl group. All such isomers, as well as mixtures thereof, are intended to be included in this invention.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease or symptoms thereof.

Isoxazoline parasiticides are a class of parasiticide agents that are conventionally used as insecticides and acaricides for veterinary indications. One or more isoxazoline parasiticides can be utilized with systems and methods as disclosed herein, either alone or in combination with other therapeutic agents. In some embodiments, patients in need thereof can be treated with an active agent from the isoxazoline parasiticide family of chemicals, which include but are not limited to isoxazoline-substituted benzamide derivatives. Not to be limited by theory, isoxazoline parasiticides can act as GABA-chloride antagonists to selectively target the nervous system of certain organisms. The GABA-mediated chloride influx can lead to hyperpolarization of the cellular membrane and generates an inhibitory postsynaptic potential, which decreases the probability of an action potential, and lead to paralysis and eventual death of the organisms. The isoxazoline parasiticide can include, for example, any number of fluralaner, sarolaner, lotilaner, afoxolaner, isocycloseram, and/or fluxametamide, including derivatives, analogues, and L- and D-isomers thereof, including but not limited to enantiomers, compositions comprising racemic mixtures, and enantiomerically pure compositions. In some embodiments, the isoxazoline parasiticide, or other active ingredients as disclosed herein are the only active ingredient utilized in the formulation and/or method. In some embodiments, the isoxazoline parasiticide is an isoxazoline-substituted benzamide derivative. In some embodiments, the isoxazoline parasiticide has one, two, three, or more fluorine groups, such as trifluorine groups in its chemical structure (e.g., R—CF₃). In some embodiments, the formulation can include a precursor compound (e.g., a isoxazole carboxylic acid, including isoxazole-4-carboxylic acid), or a degradation compound (e.g., isoxazolethiopene carboxylic acid) to other isoxazoline parasiticides, instead or, or in addition to isoxazoline parasiticides disclosed elsewhere herein, in amounts/concentrations disclosed elsewhere herein for example. In some embodiments, a formulation does not include any precursor or degradation compounds, including those disclosed herein. In some embodiments, a formulation can include pyrazole-5-carboxamides including an arylisoxazoline moiety.

In some embodiments, systems and methods can be therapeutically effective to kill a disease-carrying vector, which could require only a single oral dose of an isoxazoline parasiticide, such as less than about 500 mg for example, or in the range of about 100-1,000 mg, or about or less than about 1,000, 900, 800, 700, 600, 500, 400, 300, 200, 100, 75, 50, 25 mg, or less, or ranges including any two of the foregoing values, to provide vector protection for about or at least about 45, 60, 75, 90 days, or more. In some embodiments the dosage may only require a single oral dose of less than 100 mg, for example 50-100 mg, if it were desirable to reduce the duration or magnitude of systemic exposure. In some embodiments, multiple dosages could be available to deliver preferred blood plasma levels for individuals of differing body weight. In some embodiments the dosage may be lower, more effective, and better absorbed in the gut if given with or within 30, 60, or 90 min before or after food intake. In some embodiments, disclosed herein is providing relatively low dose administration of an isoxazoline parasiticide for vector control, including but not limited to malaria vector control. Such low dose administration can provide a very low systemic exposure for safety without necessarily requiring a high mosquito kill rate. Such formulations can methods can provide coverage that weakens (or kills a portion of) the vector (e.g., mosquitos or others) such that they are not able to bite or otherwise transmit the organism to the next person. Generally, a higher dose is needed to kill ticks than mosquitos, by, for example, 2, 3, 4, or 5-fold. In some embodiments, a formulation could include a single oral dose, followed by none, or a limited number of follow-up smaller doses (e.g., 1, 2, 3, 4, 5, 6, 7, or more or less follow-up doses (or ranges including any two of the foregoing values) administered daily, weekly, or other intervals as disclosed for example elsewhere herein). In some embodiments, the therapeutic agent is provided in a one-time low dose given at some interval (e.g., monthly or longer), including but not limited to every 2-3 weeks, 1, 2, 3, 4, 5, 6 months, or more or less, or ranges including any of the foregoing values. In some embodiments, administration once every 3-4 months could ultimately mean once per year for places with seasonal malaria transmission. In some embodiments, the formulation or method results in a peak or random blood, plasma, serum, or other fluid level in the patient of isoxazoline parasiticide, or other therapeutic agent including those disclosed elsewhere herein that is no more than about 1,000, 750, 500, 250, 200, 175, 150, 125, 100, 75, 50, 25, 20, 15, 10, 5, 4, 3, 2, 1 ng/ml or even less.

In some embodiments, administration in a therapeutically effective dose can result in sufficient systemic exposure/plasma concentration of an isoxazoline parasiticide to not only provide vector protection, but additionally disrupt the health, and/or life cycle (e.g., replication) of Plasmodium or other species, including parasiticidal activity. Such other species could include tick-borne organisms such as Borrelia Burgdorferi, Borrelia mayonii, Borrelia miyamotoi, other Borrelia species, Babesia microti, other Babesia species, Ehrlichia muris eauclairensis, Ehrlichia chaffeensis, Ehrlichia ewingii, other Ehrlichia species, Anaplasma phagocytophilum, other Anaplasma species, Francisella tularensis, other Francisella species, Rickettsia rickettsia, Rickettsia, parkeri, other Rickettsia species, Powassan virus, Heartland virus, Bourbon virus, and Colorado tick fever virus. Not to be limited by theory, this can require multiple and/or higher dosing than for vector control indications as discussed above. In some embodiments, the dosing can be more than a single oral dose over a 45, 60, 75, 90, or more day period, such as at least about 2, 3, 4, 5, 6, 7, or more doses. The doses can be, for example, 1, 2, 3, 4, 5, 6 or more times weekly, or 1, 2, 3, or more times daily, for example. In some embodiments, the cumulative dosing of isoxazoline parasiticide administered in a course of treatment can be at least about 500 mg, 1 g, 1.5 g, 2 g, 3 g, 4 g, 5 g, 6 g, 7 g, 8 g, 9 g, 10 g, or more divided over a period of at least about 1 day, 2 days, 3 days, 4 days, 5 days, 6 days, 7 days, 2 weeks, 3 weeks, 4 weeks, 45, 60, 75, 90, or more days. In some embodiments, the dosage can be more than a single oral dose, of which the dosages may be different amounts. In some embodiments, the number of Plasmodium organisms in a target location in an individual can be decreased by at least 10%, 20%, 30%, 40%, 50%, 60%, 70%, 80%, 90% or more 1, 2, 3, 4, 5, 6, 7, 10, 14, or more days after therapy relative to prior to the initiation of therapy.

In some embodiments, the therapeutic agent, e.g., isoxazoline parasiticide and/or other agents can be administered to create peak, trough, or random plasma concentrations over 1, 2, 3, 4, 5, 6, 7, or more days, for example, between about 1 ng/mL and about 50,000 ng/mL, between about 10 ng/mL and about 10,000 ng/mL, between about 100 ng/mL and about 5,000 ng/mL, about, at least about, or no more than about 1, 5, 10, 50, 100, 200, 250, 300, 350, 400, 450, 500, 600, 700, 800, 900, 1,000, 5,000, 10,000, 25,000, or 50,000 ng/mL, or ranges including any two of the foregoing values. In some embodiments, the isoxazoline parasiticide can be administered to create peak, trough, or random plasma concentrations over 1, 2, 3, 4, 5, 6, 7, or more days, for example, between about 1 nM and about 50,000 nM, between about 10 nM and about 10,000 nM, between about 100 nM and about 5,000 nM, about, at least about, or no more than about 1, 5, 10, 50, 100, 200, 250, 300, 350, 400, 450, 500, 600, 700, 800, 900, 1,000, 5,000, 10,000, 25,000, or 50,000 nM, or ranges including any two of the foregoing values.

In some embodiments, the therapeutic agent, e.g., isoxazoline parasiticide and/or other agents can be administered in such a way to create a long-acting (for example, 1, 2, 3, 4, 5, 6, 7, or more days, 2, 3, 4, or more weeks, 2, 3, 4, 5, 6, or more months, or more or less) and relatively constant blood plasma exposure. As a non-limiting example, the parasiticide could be delivered via solid oral tablets once per week over a three-week interval with no further therapy with the parasiticides within about 1, 2, 3, 4, 5, 6 months or more or less thereafter, wherein each tablet systemically delivers isooxazoline so as to maintain relatively constant blood plasma levels (e.g., variation of less than about 10%, or less than about 20%) over a period of about 1, 2, 3, 4, 5, 6 months or more or less, or ranges including any two of the foregoing values.

In some embodiments, the therapeutic agent, e.g., isoxazoline parasiticide and/or other agents can be administered in only a one-time single dose, or in about, at least about, or no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 doses, or ranges including any two of the foregoing values.

In some embodiments, a therapeutically effective dose can inhibit Plasmodium replication in a target organ of a mammal, such as a human, and improve signs and/or symptoms of malaria. The target organ can be the liver, spleen, bone marrow, or other areas.

In some embodiments, systems and methods can treat various Plasmodium species, including but not limited to P. falciparum, P. vivax, P. malaria, P. ovale, P. knowlesi, and others.

In some embodiments, the dose can result in systemic exposure/plasma concentration that is meaningfully higher than which is required to treat an individual with an infection/infestation, in order to reduce the probability of resistance evolution of the parasite.

In some embodiments, an isoxazoline parasiticide formulation can be delivered orally (e.g., in tablet, chew, capsule, syrup, sublingual, dispersible, crushable, dissolvable, or other formulation), via injection (e.g., intramuscular, subcutaneous, intravenous, intraosseus), transdermally (e.g., via a patch, cream, ointment, oil, etc.), topically without transdermal absorption (e.g., via a band-aid, film, clothing, etc.) via an oral or nasal spray, via a transrectal or transvaginal suppository, an eye drop formulation at a dose sufficient for therapeutically effective systemic bioavailability, and the like. In some embodiments, the isoxazoline parasiticide may be administered in more than one format or administration route to achieve some desirable effect, for example, as both an oral tablet and dermal application via a patch, cream, ointment, and the like.

In some embodiments, the formulation is configured for systemic use, and not local use. In some embodiments, the formulation is configured to be delivered via an ophthalmic route. In some embodiments, the formulation is configured to be delivered transdermally.

In some embodiments, an isoxazoline parasiticide agent can be utilized in combination with one, two, or more additional anti-malarial, antibiotic, and/or anti-parasitic agents for unexpectedly synergistic effect to treat other diseases or conditions, such as malaria, scabies, lice, or nematode infestation, to reduce the probability of resistance, to boost efficacy in killing or otherwise inactivating vectors, or for other beneficial effect. The additional anti-malarial agent(s) could be a different isoxazoline parasiticide agent (e.g., fluralaner and lotilaner together, via the same or different administration routes, as one non-limiting example). The additional anti-malarial agent could be one or more of, for example, chloroquine, hydroxychloroquine, coartem, mefloquine, proguanil, chlorproguanil, chlorguanide, biguanides, pyrimidine, trimethoprim, chloroquine, lumefantrine, atovaquone, pyrimethamine, pyrimethamine-sulfadoxine, pyrimethamine-dapsone, halofantrine, quinine, quinidine, cinchonine, cinchonidine, Quinimax (quinine-quinidine-cinchonin), amodiaquine, amopyroquine, sulfonamides and other sulfa agents (e.g., sulfadoxine, trimethoprim-sulfamethoxazole), artemisinin, ASAQ (artesunate-amodiaquine), arteflene, artemether, artesunate, primaquine, pyronaridine, clindamycin, and combinations thereof. The additional agent(s) could be another anti-parasitic agent, such as ivermectin, moxidectin, selamectin, doramectin, eprinomectin, abamectin, or any other of the avermectin class. In other embodiments, the isoxazoline parasiticide could be combined with an antibiotic, such as doxycycline, amoxicillin, cefuroxime axetil, azithromycin, clarithromycin, or erythromycin. In other embodiments, the isoxazoline parasiticide can be the only active agent in the systemic formulation. In other embodiments, any two of the foregoing active agents can be utilized, with or without an isoxazoline parasiticide.

Also disclosed herein are methods of simultaneously treating malaria in infected individuals and creating mass population resistance to malaria by administrating an isoxazoline parasiticide agent to both infected and uninfected individuals in a selected geography. The isoxazoline parasiticide can be at a therapeutically effective dose sufficient to treat Plasmodium infection in an infected individual and sufficient to kill mosquitos who feed on the blood of dosed individuals. The mass population dosing can be effective in treating Plasmodium in infected individuals, and also reduce the mosquito population in the geography.

In some embodiments, disclosed herein are methods of vector-borne disease treatment and/or prevention by combining an isoxazoline antiparasitic agent with vector control methods or technologies. Vector control methods or technologies could include one, two, or more of the following: nets, pesticide sprays or other formulations, education, standing water removal, traps, smoke/incense, and the like. In some embodiments, the isoxazoline antiparasitic agent may be used to coat, cover, saturate, or otherwise be administered to any item that may come in contact with a relevant vector, such as nets, indoor or outdoor walls, floors, ceilings, furniture, clothing (including shoes, boots, gloves, hats, glasses, etc), fences, railings, and the like.

In some embodiments, the administered dose of the isoxazoline parasiticide formulation is therapeutically effective to treat Plasmodium infestation in an infected individual, but not high enough as to produce/induce undesired and/or unacceptable side effects.

In some embodiments, disclosed herein is a sustained-release formulation and or drug-device configuration utilized to reduce the frequency of dosing and/or increase duration of effect and/or increase drug compliance and/or reduce the probability and/or rate of vector resistance to the isoxazoline parasiticide and/or other therapeutic agents used in a combination therapy. A long half-life isoxazoline parasiticide formulation can be utilized in combination with a slow/sustained-release technology to provide a very long period of sustained plasma drug levels. The sustained period could be, for example, about or at least about 1, 2, or 3 weeks, 1, 2, 3, 4, 5, 6, 7, 8, 9, 10, 11, 12, 15, 18, 21, 24 months, 3, 4, 5, or more years, or ranges including any two of the foregoing values. The sustained-release formulation could include any of the embodiments described herein, including a dermal patch, a cream, an ointment, a gel (including any type of hydrogel containing an isoxazoline, which could be delivered dermally, rectally, via injection, or by other route of administration depending on desired effect), oral dosage form, or any other formulation.

In some embodiments, the isoxazoline parasiticide could be administered via a formulation that causes release into systemic circulation at a specified region of the body. For example, the parasiticide could become available for systemic absorption following oral administration by absorption in the stomach, or in the gut, depending on the desired performance. Such absorption could be enhanced if given in close proximity to food intake.

In some embodiments, the isoxazoline parasiticide's effect as described in other embodiments as disclosed herein may be supported, enhanced, or otherwise improved by the activity of metabolites of the isoxazoline parasiticide.

In some embodiments, the water supply for a particular geography can be dosed with an isoxazoline parasiticide to maintain a concentration of isoxazoline parasiticide therapeutically effective to provide mass population prophylaxis.

In some embodiments, animals can be dosed with an isoxazoline parasiticide to maintain a concentration of isoxazoline parasiticide therapeutically effective to provide, support, or enhance vector control. Such animals could include livestock (for example, cattle, pigs, sheep), horses or other animals used for transportation of people and/or goods, other domesticated animals including indoor and/or outdoor pets, and non-domesticated animals such as mice, bird, and/or deer, as may be valuable in supporting further reduction of vector populations.

In some embodiments, patients could be treated with gene therapy (e.g., a viral or plasmid vector) that causes a treated individual to synthesize an isoxazoline parasiticide sufficient to cause natural malaria resistance. In some embodiments, animals including farm animals could be treated with gene therapy (e.g., a viral or plasmid vector) that causes a treated animal to synthesize an isoxazoline parasiticide that can be excreted in milk, etc. sufficient to serve as a bioreactor.

In some embodiments, a formulation and/or packaging can be specifically configured to be tolerant of extreme environmental conditions (e.g., high heat or UV light exposure, for example). The packaging could include opaque or reflective packaging, such as waterproof packaging in some cases. In some embodiments, a flavoring and/or sweetening agent can be added to an oral formulation to improve taste.

In some embodiments, an isoxazoline parasiticide formulation can be utilized to treat other indications/diseases via systems and methods as disclosed elsewhere herein. The diseases could also be spread via an insect vector, such as a mosquito vector. The disease could include, for example, dengue, West Nile virus, chikungya, yellow fever, filiarisis, tularemia, dilofilariasis, Japanese encephalitis, St. Louis encephalitis, Western equine encephalitis, Zika, and the like. In some embodiments, the disease could include, for example, EEE (Eastern Equine Encephalitis) and other tick-borne diseases/pathogens: Lyme Disease, Anaplasmosis, Ehrlichiosis, Babesiosis, Borrelia miyamotoi disease, Rickettsia parkeri spotted fever, Pacific Coast tick fever, Ehrlichia muris-like infection, Heartland virus, Bourbon virus, B. mayonii infection, and other tickborne diseases.

In some embodiments, an isoxazoline parasiticide formulation can be utilized to treat other endoparasitic conditions (or protozoan or amebic diseases), including river blindness (onchoceriasis), leishmaniasis, cryptosporidiosis, amoebiasis, Chagas disease, African trypanosomiasis, and others.

In some embodiments, not to be limited by theory, an isoxazoline parasiticide used systemically for malaria treatment or prophylaxis could include any number of the following properties: Mechanism of action can involve one, two, or more of: inhibition or activation of 5HT3 receptors, GABA Cl-channels, glutamate-gated Cl-channels, Serpentine receptors, or depolarization or other neural activity on Plasmodium species; Mechanism of action involves blocking nuclear import of the Plasmodium signal recognition particle (SRP), or involvement of the farnesoid X receptor for regulation of glucose homeostasis; Inhibits the hepatic stage of Plasmodium infection by impairing parasite development inside hepatoyctes, and reducing resulting parasitemia, thus reducing disease severity and enhancing patient survival; and/or may be used in combination with an avermectin, e.g., ivermectin and/or other therapeutic agents as discussed herein for activity against Plasmodium.

In some embodiments, pharmaceutical formulations and methods as disclosed herein can be used to treat or prevent infection by one, two, or more pathogens, and can have a direct effect on the pathogen (and not just a vector that may be harboring the pathogen). Pathogens can include, for example, any number of the following: viruses (including but not limited to coronavirus, human immunodeficiency virus, herpes simplex virus, papilloma virus, influenza virus, parainfluenza virus, hepatitis virus, Coxsackie Virus, herpes zoster virus, measles virus, mumps virus, rubella, rabies virus, hemorrhagic viral fevers, H1N1, and the like), prions, parasites, fungi, mold, yeast and bacteria (both gram-positive, gram-negative, anaerobic, acid-fast, and the like).

In some embodiments, the pathogen is a virus, e.g., a DNA or RNA virus. In some embodiments, the virus is an RNA virus, e.g., a single or double-stranded virus. In some embodiments, the RNA virus is a positive sense, single-stranded RNA virus. In some embodiments, the virus is part of the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-CoV), SARS-CoV-2 (COVID-19), Middle Eastern Respiratory Syndrome Coronavirus (MERS-CoV), human coronavirus HKU1, or human coronavirus OC43. In some embodiments, the gammacoronavirus is infectious bronchitis virus (IBV). In some embodiments, the coronavirus is an animal virus and causes feline intestinal peritonitis (FIP), canine respiratory coronavirus (CRCoV), bovine coronavirus, or equine enteric coronavirus.

The formulations can be administered to, for example, a human and/or other non-human animals such as dogs, cats, livestock, primates, bats, and the like.

Not to be limited by theory, in some embodiments, a pharmaceutical formulation, including but not limited to an isoxazoline parasiticide and/or other therapeutic agents disclosed elsewhere herein, can bind to, inhibit expression of, or otherwise directly or indirectly affect any number of:

a spike (S) glycoprotein contained on the viral surface;

the receptor binding domain (RBD) on S1 involved in transmembrane angiotensin-converting enzyme 2 (ACE2) binding;

ACE2 (angiotensin-converting enzyme 2)—a viral receptor protein on the host cells which binds to viral S protein;

the angiotensin AT2 receptor;

the S2 protein (involved in viral fusion with the cell membrane);

an envelope small membrane nucleoprotein (E protein);

a membrane protein (N protein);

3CLpro (coronavirus main protease 3CLpro) and/or PLpro (papain-like protease PLpro)—proteases for the proteolysis of viral polyprotein into functional units);

RdRp (RNA-dependent RNA polymerase for replicating the viral genome);

TMPRSS2 (transmembrane protein, serine 2—a host cell-produced protease that primes S protein to facilitate its binding to ACE2) and/or

hemagglutinin esterase (HE), in order to treat or prevent viral infections.

In some embodiments, a formulation, including but not limited to an isoxazoline parasiticide (e.g., for example, fluralaner, sarolaner, lotilaner, afoxolaner, fluxametamide, and isocycloseram) and/or other therapeutic agents disclosed elsewhere herein, can be used as a single active agent, or have unexpectedly synergistic effects when utilized in combination with additional agents, to treat a pathogenic infection, such as a viral infection, including but not limited to SARS-CoV 2 (COVID 19) and others. In some embodiments, other forms including those disclosed herein, such as, for example, derivatives, analogues, and L- and D-isomers thereof, including but not limited to enantiomers, compositions comprising racemic mixtures, and enantiomerically pure compositions can also be utilized.

In some embodiments, a formulation, including but not limited to a spinosyn and/or other therapeutic agents disclosed elsewhere herein, can be used as a single active agent, or have unexpectedly synergistic effects when utilized in combination with additional agents to treat a pathogenic infection, such as a viral infection, including but not limited to SARS-CoV 2 (COVID 19) and others. The spinosyn could include, for example, spinosyn A, B, C, D, E, F, G, H, J, K, L, M, N, O, P, Q, R, S, T, U, V, W, Y, and the like. Spinosyns are a family of macrocyclic lactones having pesticidal activity on a variety of pests. The early identified spinosyns were found to have a 5,6,5-tricylic ring system, fused to a 12-membered macrocyclic lactone, a neutral sugar (rhamnose), and an amino sugar (forosamine). Spinosyns are also disclosed in U.S. Pat. Nos. 5,496,931, 5,670,364, 5,591,606, 5,571,901, 5,202,242, 5,767,253, 5,840,861, 5,670,486 and 5,631,155, as well as U.S. Pub. No. 2020/0031859 to Santos et al., each of which are hereby incorporated by reference in their entireties. In some embodiments, other forms including those disclosed herein, such as, for example, derivatives, analogues, and L- and D-isomers thereof, including but not limited to enantiomers, compositions comprising racemic mixtures, and enantiomerically pure compositions can also be utilized.

In some embodiments, a formulation, including but not limited to albendazole, cambendazole, fenbendazole, flubeiidazole, mebendazole, oxfendazole, parabendazole, tiabendazole, triclabendazole, amitraz, demiditraz, clorsulon, closantel, oxyclonazide, rafoxanide, cyphenothrin, flumethrin, permethrin, promazine, derquantel, diamphenetide, dicycianil, dinotefuran, imidacloprid, nitenpyram, thiamethoxam, abamectin, doramectin, emamectin, epnnomectin, ivermectin, moxidectin, selamectin, milbemycin oxime, emodepside, epsiprantel, fipronil, fluazuron, fluhexafon, indoxacarb, levamisol, lufenuron, metaflumizone, methoprene, monepantel, morantel, niclosamide, nitroscanate, nitroxynii, novaluron, oxantel, praziquantel, pyrantel, pynprole, pvriproxyfen, sisaproml, spinosad, spinetoram, lindane, picrotoxin, dieldrin, alpha-endosulfan, and/or triflumezopyrim can be used as a single active agent, or have unexpectedly synergistic effects when utilized in combination with additional agents to treat a pathogenic infection, such as a viral infection, including but not limited to SARS-CoV 2 (COVID 19) and others as disclosed elsewhere herein.

In some embodiments, a formulation, including but not limited to a meta-diamide (e.g., broflanilide, tetraniliprole, or cyclaniliprole), a cyclodiene, and/or a macrocyclic lactone (including avermectins and milbemycin); an Alzheimer's disease drug can be the active agent, such as galantamine, donepezil and other piperidine analogues, rivastigmine and other carbamate analogues, tacrine, 7-methoxytacrine, other pyridine analogues, huperazine A and other alkaloid analogues can be used as a single active agent, or have unexpectedly synergistic effects when utilized in combination with additional agents to treat a pathogenic infection, such as a viral infection, including but not limited to SARS-CoV 2 (COVID 19) and others.

In some embodiments, a formulation, including but not limited to a formamidine parasiticides can be used as a single active agent, or have unexpectedly synergistic effects when utilized in combination with additional agents to treat a pathogenic infection, such as a viral infection, including but not limited to SARS-CoV 2 (COVID 19) and others. A formamidine parasiticide can be, for example, amitraz. N-(2,4-Dimethylphenyl)-N-methyformamidine (DPMF), a metabolite of amitraz can be another active therapeutic agent, alone or in addition. 2,4-dimethylanaline is a hydrolysis metabolite of DPMF and can also be an active therapeutic agent in other embodiments. In some embodiments, other forms including those disclosed herein, such as, for example, derivatives, analogues, and L- and D-isomers thereof, including but not limited to enantiomers, compositions comprising racemic mixtures, and enantiomerically pure compositions can also be utilized.

In some embodiments, a formulation, including but not limited to a formamidine parasiticides can be used as a single active agent, or have unexpectedly synergistic effects when utilized in combination with additional agents to treat a pathogenic infection, such as a viral infection, including but not limited to SARS-CoV 2 (COVID 19) and others. The chemical structures of these insecticides are characterized by a central pyrazole ring with a phenyl group attached to one of the nitrogen atoms of the pyrazole. Some non-limiting examples of phenyl pyrazole parasiticides include, for example, acetoprole, ethiprole, fipronil, flufiprole, pyraclofos, pyraflprole, pyriprole, pyrolan, and vaniliprole.

In some embodiments, a formulation, including but not limited to organophosphates, can be used as a single active agent, or have unexpectedly synergistic effects when utilized in combination with additional agents to treat a pathogenic infection, such as a viral infection, including but not limited to SARS-CoV 2 (COVID 19) and others. The organophosphate could include one or more of, for example, acephate, azamethiphos, azinphos ethyl, azinphos methyl, bromophos, bromophos ethyl, cadusofos, carbophenythion, chlormephos, chlorphoxim, chlorpyrifos, chlorpyrifos-methyl, chlorthiophos, chlorvinophos, croumaphos, crotoxyphos, crufomate, cyanofenphos, cyanophos, demephron-O, demephron-S, demeton-O, demeton-S, demeton-S-methyl, demeton-S-methylsulphon, dialifos, diazinon, dichlofenthion, dichlorvos, dicrotophos, dimefphox, dimethoate, dioxabenzophos, dioxathion, disulfoton, ditalmifos, edifenphos, EPBP, EPN, ESP, ethion, ethopropos, etrimfos, famphur, fenamiphos, fenchlorphos, fenitrothion, fensulfothion, fenthion, fenofos, formothion, fosmethilan, heptenophos, isazofos, isofenphos, isothioate, isoxathion, jodfenphos, leptophos, metrifonate, malathion, menazon, mephosfolan, methacrifos, methamidophos, methidathion, mevinphos, monocrotophos, naled, omethoate, oxydemeton-methyl, parathion, parathion-methyl, phenthoate, phorate, phosalone, phosmet, phosphamidon, phosphamidon amide, phospholan, phoxim, pirimiphos-ethyl, pirimiphos-methyl, profenofos, propaphos, propetamphos, prothiofos, prothoate, pyraclofos, pyridaphenthion, quinlphos, schradan, sulfotep, sulprofos, temephos, TEPP, terbufos, tetrachlorvinphos, thiometon, thionazin, triazophos, trichlorfon, vamidothion, a prodrug of these and a pharmaceutically acceptable salt or ester of these. In some embodiments, the organophosphate can be dichlorvos or a prodrug or pharmaceutically acceptable salt or ester thereof. In some embodiments, the organophosphate can be metrifonate or a prodrug or pharmaceutically acceptable salt or ester thereof. In some embodiments, other forms including those disclosed herein, such as, for example, derivatives, analogues, and L- and D-isomers thereof, including but not limited to enantiomers, compositions comprising racemic mixtures, and enantiomerically pure compositions can also be utilized.

The additional agent could be, for example, other agents with anti-viral activity, including but not limited to baricitinib or other JAK inhibitors; lopinavir and/or ritonavir, darunavir, favipiravir, remdesivir, ribavirin, galidseivir, BCX-4430 (salt form of galidesivir), Arbidol, chloroquine, hydroxychloroquine, mefloquine, nitazoxanide, acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, rimantadine, oseltamivir, zanamivir, and/or baloxavir, as well as other agents as disclosed elsewhere herein.

In some embodiments, the therapeutic agent is provided in a single, one-time low dose given at some interval (e.g., monthly or longer), including but not limited to every 2-3 weeks, 1, 2, 3, 4, 5, 6 months, or more or less, or ranges including any of the foregoing values. In some embodiments, administration once every 3-4 months could ultimately mean once per year for places with seasonal pathogen transmission. In some embodiments, the formulation or method results in a peak or random blood, plasma, serum, or other fluid level in the patient of isoxazoline parasiticide, or other therapeutic agent including those disclosed elsewhere herein that is no more than about 1,000, 750, 500, 250, 200, 175, 150, 125, 100, 75, 50, 25, 20, 15, 10, 5, 4, 3, 2, 1 ng/ml or even less. In some embodiments, a single dose, or multiple doses can be provided, each dose of the formulation with a half-life of about or at least about 20, 25, 30, 35, 40, 45, 50, 55, 60, or more days. In some cases, such long-acting dosing can have several advantages over alternative potential drugs for transmission prophylaxis or prolonged treatment effect, decreasing viral loads or preventing secondary complications such as pneumonia, acute respiratory distress syndrome (ARDS), septic shock, cardiomyopathy, renal failure, etc; given the relatively long half life. However, in some embodiments, dosing could be, for example, about or at least about 1, 2, 3, 4, 5, 6, 7, 8, or more times daily, such as 1 to 2 times daily. In some embodiments, therapy could also be weekly, single dose or a limited-course of treatment.

In some embodiments, the dosing can be more than a single oral dose over a 45, 60, 75, 90, or more day period, such as at least about 2, 3, 4, 5, 6, 7, or more doses. The doses can be, for example, 1, 2, 3, 4, 5, 6 or more times weekly, or 1, 2, 3, or more times daily, for example. In some embodiments, the cumulative dosing of an active agent, such as for example, an isoxazoline parasiticides or any other agent as disclosed herein administered in a course of treatment can be about, at least about, or no more than about 50 mg, 100 mg, 200 mg, 300 mg, 400 mg, 500 mg, 1 g, 1.5 g, 2 g, 3 g, 4 g, 5 g, 6 g, 7 g, 8 g, 9 g, 10 g, or more divided over a period of at least about 1 day, 2 days, 3 days, 4 days, 5 days, 6 days, 7 days, 2 weeks, 3 weeks, 4 weeks, 45, 60, 75, 90, or more days. In some embodiments, the dosage can be more than a single oral dose, of which the dosages may be different amounts. In some embodiments, the viral load of an individual can be decreased by at least 10%, 20%, 30%, 40%, 50%, 60%, 70%, 80%, 90% or more, 1, 2, 3, 4, 5, 6, 7, 10, 14, or more days after therapy relative to prior to the initiation of therapy.

In some embodiments, an active agent, such as for example, an isoxazoline parasiticides or any other agent or combination of agents as disclosed herein can be administered to create peak, trough, or random plasma concentrations over 1, 2, 3, 4, 5, 6, 7, or more days, for example, between about 1 ng/mL and about 50,000 ng/mL, between about 10 ng/mL and about 10,000 ng/mL, between about 100 ng/mL and about 5,000 ng/mL, about, at least about, or no more than about 1, 5, 10, 50, 100, 500, 1,000, 5,000, 10,000, 25,000, or 50,000 ng/mL, or ranges including any two of the foregoing values. In some embodiments, the isoxazoline parasiticide can be administered to create peak, trough, or random plasma concentrations over 1, 2, 3, 4, 5, 6, 7, or more days, for example, between about 1 nM and about 50,000 nM, between about 10 nM and about 10,000 nM, between about 100 nM and about 5,000 nM, about, at least about, or no more than about 1, 5, 10, 50, 100, 200, 250, 300, 350, 400, 450, 500, 600, 700, 800, 900, 1,000, 5,000, 10,000, 25,000, or 50,000 nM, or ranges including any two of the foregoing values. In some embodiments, the active agent, such as, for example, an isoxazoline parasiticide can be administered in such a way to create a long-acting (for example, 1, 2, 3, 4, 5, 6, 7, or more days, 2, 3, 4, or more weeks, 2, 3, 4, 5, 6, or more months, or more or less) and relatively constant blood plasma exposure. As a non-limiting example, the active agent, e.g, isoxazoline parasiticide could be delivered via solid oral tablets once per week over a three-week interval with no further therapy with the parasiticides within about 1, 2, 3, 4, 5, 6 months or more or less thereafter, wherein each tablet systemically delivers isooxazoline so as to maintain relatively constant blood plasma levels (e.g., variation of less than about 10%, or less than about 20%) over a period of about 1, 2, 3, 4, 5, 6 months or more or less, or ranges including any two of the foregoing values.

In some embodiments, an active agent, for example, an isoxazoline parasiticide formulation or any other agent or combination of agents as disclosed herein can be delivered orally (e.g., in tablet, chew, capsule, syrup, sublingual, dispersible, crushable, dissolvable, or other formulation), via injection (e.g., intramuscular, subcutaneous, intravenous, intraosseus), transdermally (e.g., via a patch, cream, ointment, oil, etc.), via an oral or nasal spray, via a transrectal or transvaginal suppository, an eye drop formulation at a dose sufficient for therapeutically effective systemic bioavailability, and the like. In some embodiments, the isoxazoline parasiticide may be administered in more than one format or administration route to achieve some desirable effect, for example, as both an oral tablet and dermal application.

In some embodiments, an active agent, for example, an isoxazoline parasiticide formulation or any other agent or combination of agents as disclosed herein can be given in individual doses of, for example, about, at least about, or no more than about 1 mg, 2 mg, 3 mg, 4 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1250 mg, 1500 mg, 1750 mg, 2000 mg, 2500 mg, 3000 mg, 4000 mg, 5000 mg, or more or less, or ranges including any two of the foregoing values. The dosing could be at least about, about, or no more than about 1, 2, or 3 times a day; every other day, every third day, 1, 2, 3, 4, 5, 6, or 7 times a week; once every 2 weeks, once a month, once every two months, once every three months, a single one-time dose only, or ranges including any two of the foregoing values. The therapeutic regimen can be administered for a total of about, at least about, or no more than about 1, 2, 3, 4, 5, 6, 7, 8, 9, 10, 11, 12, 13, 14, 21, 28 days, or more or less, or ranges including any two of the foregoing values.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “orally administering an isoxazoline parasiticide pharamaceutical formulation” includes “instructing the oral administration of an isoxazoline parasiticide pharmaceutical formulation.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A method of treating malaria, comprising: administering a therapeutic dose of an isoxazoline parasiticide formulation therapeutically effective to an individual in need thereof, the therapeutic dose sufficient to be systemically bioavailable sufficient to inhibit the health or life cycle of a Plasmodium species in the individual.

2. The method of claim 1, comprising administering a plurality of doses of the isoxazoline parasiticide formulation within a 30 day period.

3. The method of claim 1, wherein the formulation is administered orally.

4. The method of claim 1, wherein the formulation is administered parenterally.

5. The method of claim 1, wherein the formulation is administered transdermally.

6. The method of claim 1, wherein the Plasmodium species is selected from the group consisting of: P. falciparum, P. vivax, P. malaria, P. ovale, and P. knowlesi.

7. The method of claim 1, wherein the formulation is therapeutically effective to inhibit the health or life cycle of the Plasmodium species in a liver of the individual.

8. The method of claim 1, further comprising administering another therapeutic agent therapeutically effective to inhibit the health or life cycle of a Plasmodium species in the individual.

9. The method of claim 8, wherein administering another therapeutic agent occurs in the same formulation as the isoxazoline parasiticide formulation.

10. The method of claim 1, further comprising identifying the individual diagnosed with malaria.

11. The method of claim 1, wherein the isoxazoline parasiticide is selected from the group consisting of fluralaner, lotilaner, sarolaner, and afoxolaner.

12. An isoxazoline parasiticide formulation for use in treating malaria, said formulation therapeutically effective to an individual in need thereof, said formulation sufficient to be systemically bioavailable sufficient to inhibit the health or life cycle of a Plasmodium species in the individual.

13. The method of claim 12, wherein the isoxazoline parasiticide is selected from the group consisting of fluralaner, lotilaner, sarolaner, and afoxolaner.

14. A method of prophylaxis against a vector-borne disease, comprising: administering a single therapeutic dose of an isoxazoline parasiticide formulation therapeutically effective to an individual in need thereof, the single therapeutic dose sufficient to be systemically bioavailable sufficient to inhibit the health or life cycle of a vector or vector-borne disease organism for at least about 1 month.

15. The method of claim 14, sufficient to be systemically bioavailable sufficient to inhibit the health or life cycle of a vector or vector-borne disease organism for at least about 3 months.

16. The method of claim 14, wherein the isoxazoline parasiticide is selected from the group consisting of fluralaner, lotilaner, sarolaner, and afoxolaner.

17. A method of prophylaxis against a vector-borne disease, comprising: administering a plurality of spaced-apart therapeutic doses of an isoxazoline parasiticide formulation therapeutically effective to an individual in need thereof, wherein the spaced-apart therapeutic doses include between 2-7 doses within a week, but no further doses within at least about a 1 month period, the plurality of space-apart therapeutic doses sufficient to be systemically bioavailable sufficient to inhibit the health or life cycle of a vector or vector-borne disease organism for at least about 1 month.

18. The method of claim 17, wherein the plurality of spaced-apart therapeutic doses are oral doses of an isoxazoline parasiticides, the oral doses each about or less than about 500 mg.

19. The method of claim 17, comprising no further doses within at least about a 3 month period.

20. The method of claim 17, sufficient to be systemically bioavailable sufficient to inhibit the health or life cycle of a vector or vector-borne disease organism for at least about 3 months.

21. The method of claim 17, wherein the vector-borne disease comprises malaria.

22. The method of claim 17, wherein the vector-borne disease comprises Lyme disease.

23. The method of claim 17, wherein the vector-borne disease comprises one or more of the group consisting of: dengue, West Nile virus, chikungya, yellow fever, filiarisis, tularemia, dilofilariasis, Japanese encephalitis, St. Louis encephalitis, Western equine encephalitis, Zika, EEE (Eastern Equine Encephalitis), Lyme Disease, Anaplasmosis, Ehrlichiosis, Babesiosis, Borrelia miyamotoi disease, Rickettsia parkeri spotted fever, Pacific Coast tick fever, Ehrlichia muris-like infection, Heartland virus, Bourbon virus, B. mayonii infection, and other tickborne diseases.

24. A method of treating or preventing a viral infection, comprising:
administering to a subject in need thereof a pharmaceutical composition comprising an isoxazoline parasiticide, the formulation therapeutically effective to treat or prevent the viral infection in the subject.

25. The method of claim 24, comprising treating the viral infection.

26. The method of claim 24, wherein the pharmaceutical composition is a single one-time dose.

27. The method of claim 24, wherein the viral infection comprises a coronavirus infection.

28. The method of claim 24, wherein the viral infection comprises SARS-CoV 2 (COVID 19).

29. The method of claim 24, wherein the pharmaceutical composition is sufficient to be systemically bioavailable sufficient to inhibit the health or life cycle of the virus for at least about 1 month.

30. The method of claim 24, wherein the isoxazoline parasiticide is selected from the group consisting of: fluralaner, sarolaner, lotilaner, afoxolaner, fluxametamide, and isocycloseram.

31. The method of claim 24, wherein the isoxazoline parasiticide is the single active agent in the pharmaceutical composition.

32. The method of claim 24, further comprising one or more of the following additional active agents: baricitinib; lopinavir and/or ritonavir, darunavir, favipiravir, remdesivir, ribavirin, galidseivir, BCX-4430, Arbidol, chloroquine, hydroxychloroquine, mefloquine, and/or nitazoxanide.

33. A method of prophylaxis against a viral infection, comprising: administering a plurality of spaced-apart therapeutic doses of an isoxazoline parasiticide formulation therapeutically effective to an individual in need thereof, wherein the spaced-apart therapeutic doses include between 2-7 doses within a week, but no further doses within at least about a 1 month period, the plurality of space-apart therapeutic doses sufficient to be systemically bioavailable sufficient to inhibit the life cycle and/or replication of a virus for at least about 1 month.

34. The method of claim 33, wherein the viral infection comprises a coronavirus.

35. The method of claim 33, wherein the viral infection comprises SARS-CoV 2 (COVID 19).

36. The method of claim 33, wherein the plurality of spaced-apart therapeutic doses are oral doses of an isoxazoline parasiticides, the oral doses each about or less than about 500 mg.

37. The method of claim 33, comprising no further doses within at least about a 3 month period.

38. The method of claim 33, sufficient to be systemically bioavailable sufficient to inhibit the replication or life cycle of a virus for at least about 3 months.

39. The method of claim 33, wherein the isoxazoline parasiticide is selected from the group consisting of: fluralaner, sarolaner, lotilaner, afoxolaner, fluxametamide, and isocycloseram.

40. The method of claim 33, further comprising one or more of the following additional active agents: baricitinib; lopinavir and/or ritonavir, darunavir, favipiravir, remdesivir, ribavirin, galidseivir, BCX-4430, Arbidol, chloroquine, hydroxychloroquine, mefloquine, and/or nitazoxanide.

41. An isoxazoline parasiticide medicament for use in treating or preventing a pathogen, said medicament therapeutically effective to an individual in need thereof, said formulation sufficient to be systemically bioavailable sufficient to inhibit the health or life cycle of the pathogen.

42. The medicament of claim 41, wherein the pathogen comprises a virus.

43. The medicament of claim 42, wherein the virus comprises a coronavirus.

44. The medicament of claim 43, wherein the virus comprises SARS-CoV 2 (COVID 19).

45. The medicament of claim 41, for treating the pathogen.

46. The medicament of claim 41, for preventing the pathogen.

47. The medicament of claim 41, wherein the isoxazoline parasiticide is selected from the group consisting of: fluralaner, sarolaner, lotilaner, afoxolaner, fluxametamide, and isocycloseram.
